(19) United States
(12) Patent Application Publication
(10) Pub. No.: US 2009/0005683 A1
(43) Pub. Date: Jan. 1, 2009

(54) ULTRASONIC IMAGING APPARATUS AND ULTRASOUND IMAGING METHOD

(76) Inventors: Hiroshi Hashimoto, Tokyo (JP); Sei Kato, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(21) Appl. No.: 12/145,358

(22) Filed: Jun. 24, 2008

(30) Foreign Application Priority Data
Jun. 26, 2007 (JP) 2007-167841

(51) Int. Cl. A61B 8/13

(52) U.S. Cl. 600/443; 600/458

(57) ABSTRACT

An ultrasonic imaging apparatus includes a probe that transmits an ultrasonic pulse to an object to be detected and receives a reflection ultrasonic pulse train from the object to be detected, a sound pressure amplitude control device for automatically changing, with one frame including a repetition of the transmission and the reception which acquires a piece of tomographic image information of the object to be detected as a minimum change unit, a sound amplitude of the ultrasonic pulse according to an acquisition number of a frame indicative of an order of the acquisition, and a display unit that displays the tomographic image information.

### CROSS REFERENCE TO RELATED APPLICATIONS

This application claims the benefit of Japanese Patent Application No. 2007-167841 filed Jun. 26, 2007, which is hereby incorporated by reference in its entirety.

### BACKGROUND OF THE INVENTION

The subject matter disclosed herein relates to an ultrasonic imaging apparatus that gives an object to be detected a contrast agent to create a tomographic image of the object to be detected including an image of the contrast agent.

In recent years, inspection using the contrast agent is conducted in the field of the ultrasonic imaging apparatus. The contrast agent is a solution containing air bubbles of several microns (μ) order, and strongly reflects ultrasonic pulses in the object to be detected.

The contrast agent has a characteristic that an aspect is different according to the sound pressure of an irradiated ultrasonic pulse. For example, the gas bubble of the contrast agent is enlarged or reduced, or destroyed according to the magnitude of the sound pressure. An operator manually adjusts the amplitude of a drive voltage waveform that generates the ultrasonic pulse according to a diagnosis object, and conducts imaging utilizing the above features of the contrast agent (for example, refer to Japanese Unexamined Patent Publication No. 2005-074084).

On the other hand, there is being known that the sound pressure of the ultrasonic pulse that causes the features such as the enlargement/reduction motion or the destruction that are exhibited by the contrast agent is slightly changed according to the environment where the contrast agent is positioned. Then, the change is becoming one parameter indicative of a state of the environment where the contrast agent is positioned.

However, according to the background art, it is not easy to detect the sound pressure of the ultrasonic pulse that causes the enlargement/reduction motion or the destruction of the contrast agent at each of the positions where the contrast agent exists. That is, the sound pressure of the ultrasonic pulse is set in advance or manually adjusted before imaging. That the setting or adjustment is changed at each of the positions where the contrast agent exists within the imaging section takes a lot of troubles, and also the contrast agent is circulated within the object to be detected, and momentarily changes its position. As a result, the imaging timing is accompanied by difficulty.

From the above viewpoint, it is important to realize the ultrasonic imaging apparatus that is capable of readily detecting the sound pressure of the ultrasonic pulse that causes the enlargement/reduction motion or the destruction of the contrast agent over the entire region of the imaging section.

### BRIEF DESCRIPTION OF THE INVENTION

It is desirable that the problem described previously is solved.

According to a first aspect of the invention, there is provided an ultrasonic imaging apparatus, including: a probe that transmits an ultrasonic pulse to an object to be detected and receives a reflection ultrasonic pulse train from the object to be detected; a sound pressure amplitude control device for automatically changing, with one frame including a repetition of the transmission and the reception which acquires a piece of tomographic image information of the object to be detected as a minimum change unit, a sound amplitude of the ultrasonic pulse according to an acquisition number of a frame indicative of an order of the acquisition; and a display unit that displays the tomographic image information.

In the first aspect of the invention, the sound pressure amplitude of the ultrasonic pulse is changed according to the acquisition number of the frame, and the sound pressure change of the luminance value that is caused by the contrast agent is obtained in all the regions of the tomographic image.

According to a second aspect of the invention, there is provided the ultrasonic imaging apparatus according to the first aspect of the invention, wherein the object to be detected is given a contrast agent.

In the second aspect of the invention, the information related to a bloodstream of the object to be detected is collected.

According to a third aspect of the invention, there is provided the ultrasonic imaging apparatus according to the first or second aspect, wherein the sound pressure amplitude control device changes the sound pressure amplitude in proportion to the acquisition number of the frame.

In the third aspect of the invention, the amplitude of the sound pressure amplitude is uniformly changed with a time.

According to a fourth aspect of the invention, there is provided the ultrasonic imaging apparatus according to any one of first to third aspects, wherein the sound pressure amplitude control device includes: a transmission/reception unit that forms a drive voltage waveform which generates the ultrasonic pulse in a piezoelectric element of the probe; and an amplitude voltage control device for automatically changing the amplitude voltage of the drive voltage waveform according to the acquisition number.

In the fourth aspect of the invention, the sound pressure amplitude of the ultrasonic pulse is changed according to the amplitude voltage of the drive voltage waveform.

According to a fifth aspect of the invention, there is provided the ultrasonic imaging apparatus according to any one of first to fourth aspects, further including an image memory that records the tomographic image information.

In the fifth aspect of the invention, the tomographic image information is again referred to after the tomographic image information has been acquired.

According to a sixth aspect of the invention, there is provided the ultrasonic imaging apparatus according to the fourth or fifth aspect, further including an input unit that inputs the control information of the amplitude voltage control device.

In the sixth aspect of the invention, the amplitude voltage control device manually controls the change in the amplitude voltage.

According to a seventh aspect of the invention, there is provided the ultrasonic imaging apparatus according to the sixth aspect, wherein the input unit includes a start timing key that starts a change in the amplitude voltage.

In the seventh aspect of the invention, the operator starts the change in the amplitude voltage while referring to the tomographic image that is displayed on the ultrasonic imaging apparatus.

Also, according to an eighth aspect of the invention, there is provided the ultrasonic imaging apparatus according to the sixth aspect, wherein the input unit includes an administration timing key that inputs an administration timing when giving the object to be detected the contrast agent, and a delay time input key that inputs a delay time from the administration timing.

In the eighth aspect of the invention, the operator starts the change in the amplitude voltage while referring to the tomographic image that is displayed on the ultrasonic imaging apparatus.

Also, according to a ninth aspect of the invention, there is provided the ultrasonic imaging apparatus according to the eighth aspect, wherein the amplitude voltage control device starts the change in the amplitude voltage at a time delayed by the delay time from the administration timing.

In the ninth aspect of the invention, the change in the amplitude voltage starts a designated time after the contrast agent is given.

Also, according to a tenth aspect of the invention, there is provided the ultrasonic imaging apparatus according to any one of the fourth to ninth aspects, wherein the amplitude voltage control device changes the magnitude of the amplitude voltage in an increasing direction from a value of zero.

In the tenth aspect of the invention, a resonance sound pressure region of the contrast agent and the amplitude voltage of the breakdown sound pressure are sequentially detected.

Also, according to an eleventh aspect of the invention, there is provided the ultrasonic imaging apparatus according to any one of the first to tenth aspects, further including an arithmetic processing unit that arithmetically processes the tomographic image information.

In the eleventh aspect of the invention, the sound pressure characteristic of the contrast agent is calculated on the basis of the tomographic image information by the arithmetic processing unit.

Also, according to a twelfth aspect of the invention, there is provided the ultrasonic imaging apparatus according to the eleventh aspect, wherein the arithmetic processing unit includes a luminance change curve acquiring device for obtaining a luminance change curve indicative of a change in an average luminance value of a detection region due to the frame in each of the detection regions that section the tomographic image of the frame by using a plurality of frames on the same section which are different in the amplitude voltage of the drive voltage waveform from each other.

In the twelfth aspect of the invention, the luminance change curve that exhibits a sound pressure dependency characteristic of the contrast agent is obtained by the luminance change curve device in each of the detection regions.

Also, according to a thirteenth aspect of the invention, there is provided the ultrasonic imaging apparatus according to the twelfth aspect, wherein the arithmetic processing unit includes a first characteristic extracting device for obtaining a frame number of the average luminance value having a threshold value when the average luminance value of the luminance change curve passes through the threshold value.

In the thirteenth aspect of the invention, the frame number where the contrast agent is in the resonance sound pressure region.

Also, according to a fourteenth aspect of the invention, there is provided the ultrasonic imaging apparatus according to the twelfth or thirteenth aspect, wherein the arithmetic processing unit includes a second characteristic extracting device for obtaining a frame number of a maximum value of the luminance change curve.

In the fourteenth aspect of the invention, the frame number where the contrast agent is in a destruction sound pressure is obtained by the maximum value of the luminance change curve.

Also, according to a fifteenth aspect of the invention, there is provided the ultrasonic imaging apparatus according to the thirteenth or fourteenth aspect, wherein the arithmetic processing unit includes a color display device having a hue table that relates the frame number to the hue, for obtaining the hue of the detection region on the basis of the hue table.

In the fifteenth aspect of the invention, the resonance sound pressure region or the distribution of the breakdown sound pressure within the tomographic image is related to the hue so as to be visually easily recognized.

Also, according to a sixteenth aspect of the invention, there is provided the ultrasonic imaging apparatus according to the fifteenth aspect, wherein the display unit displays a color image displayed with a color of the hue in the detection region of the tomographic image.

In the sixteenth aspect of the invention, the resonance sound pressure region or the distribution of the breakdown sound pressure within the tomographic image is visualized as a color image.

Also, according to a seventeenth aspect of the invention, there is provided the ultrasonic imaging apparatus according to the eleventh aspect, wherein the arithmetic processing unit includes a capture device for obtaining the maximum value of pixel values at the same pixel position of the tomographic images of the plurality of frames by using the plurality of frames on the same section which are different in the amplitude voltage of the drive voltage waveform from each other to thereby synthesize maximum projection value image information that is comprised of the maximum value.

In the seventh aspect of the invention, the time change in the tomographic image is synthesized in a piece of image.

Also, according to an eighteenth aspect of the invention, there is provided the ultrasonic imaging apparatus according to the seventeenth aspect, wherein the display unit displays the maximum projection value image information.

In the eighteenth aspect of the invention, the time change of the tomographic image is easily grasped by the maximum projection value image information.

According to the invention, since the resonance sound pressure region of the contrast agent or the sound pressure amplitude of the breakdown sound pressure is obtained in each of the inspection regions that section the tomographic image, it is possible to recognize the resonance sound pressure region and the change of the breakdown sound pressure within the tomographic image. Also, since those inspection regions are displayed in color by the hue corresponding to the obtained sound pressure amplitude, the recognition of the change is further facilitated, and it is possible to facilitate a diagnosis of disease which changes the resonance sound pressure region of the contrast agent or the sound pressure amplitude of the breakdown sound pressure.

Further objects and advantages of the present invention will be apparent from the following description of the preferred embodiments of the invention as illustrated in the accompanying drawings.

### BRIEF DESCRIPTION OF THE DRAWINGS

FIG. 1 is a block diagram showing the entire configuration of an ultrasonic imaging apparatus.

FIG. 2 is a block diagram showing the configuration of a transmission/reception unit according to an embodiment.

FIG. 3 is an explanatory diagram showing an example of a drive voltage waveform according to the embodiment.

FIG. 4 is a block diagram showing the configuration of a control unit according to the embodiment.

FIG. 5 is an explanatory diagram showing the amplitude of the drive voltage waveform that changes in each of frames.

FIG. 6 is a flow chart showing the operation of the ultrasonic imaging apparatus according to the embodiment.

FIGS. 7(A), 7(B), and 7(C) are explanatory diagrams schematically showing a change in the B mode image according to a change in the amplitude.

FIG. 8 is an explanatory diagram showing the B mode image group that is acquired by changing the amplitude.

FIG. 9 is an explanatory diagram showing a luminance change curve of an inspection region.

FIG. 10 is an explanatory diagram showing an example of a table that relates average luminance values to hues.

FIG. 11 is an explanatory diagram schematically showing a color image that displays the distribution of the breakdown sound pressure of the contrast agent with colors.

FIG. 12 is an explanatory diagram schematically showing an example of a capture image in the case where the amplitude voltage of the drive voltage waveform changes in synchronization with the capture.

### DETAILED DESCRIPTION OF THE INVENTION

Hereinafter, a description will be given of a best mode for carrying out an ultrasonic imaging apparatus according to the invention with reference to the attached drawings. The invention is not limited by the best mode,

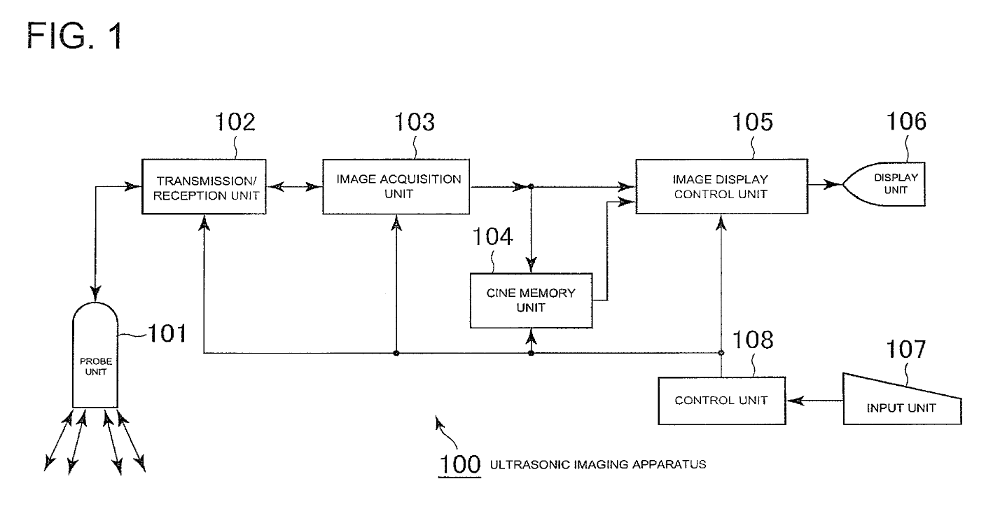

First, a description will be given of the entire configuration of an ultrasonic imaging apparatus 100 according to this embodiment. FIG. 1 is a block diagram showing the entire configuration of the ultrasonic imaging apparatus 100 according to this embodiment. The ultrasonic imaging apparatus 100 includes a probe unit 101, a transmission/reception unit 102, an image acquisition unit 103, a cine memory unit 104, an image display control unit 105, a display unit 106, an input unit 107, and a control unit 108. In this example, the transmission/reception unit 102 and the control unit 108 constitute sound pressure amplitude control means.

In the probe unit 101, piezoelectric elements are arranged in an array, the ultrasonic waves is applied to a part for transmitting or receiving the ultrasonic pulses, that is, to the object to be detected, and the reflection ultrasonic pulse train that is reflected from the interior of the object to be detected in each case is received as a time series acoustic ray. The probe unit 101 conducts electronic scanning while sequentially changing over the piezoelectric elements to be driven by means of a built-in analog multiplexer.

The transmission/reception unit 102 is connected to the probe unit 101 by means of a coaxial cable. The transmission/reception unit 102 includes a pulser that generates an electric signal of a high voltage for driving the piezoelectric element of the probe 101, and an amplifier that conducts an initial amplification of the received reflection ultrasonic pulse train. The transmission/reception unit 102 has plural pulsers and plural amplifiers that are driven substantially at the same time for conducting electronic focus. The configuration of the transmission/reception unit 102 will be described in more detail later.

The image acquisition unit 103 conducts a process for generating in a real time a B mode image or a Doppler image from the reflection ultrasonic pulse train that has been amplified by the transmission/reception unit 102. The specific processing contents are a delay addition process of a received reflection ultrasonic pulse trains an A/D (analog/digital) conversion process, and a process for writing the digital information that has been converted in the display control unit 105 or the cine memory unit 104.

The cine memory unit 104 is an image memory that saves the B mode image information that has been generated by the image acquisition unit 103.

The image display control unit 105 conducts the display frame rate conversion, control of the configuration of the image display, and the positional control, on the B mode image information that is generated by the image acquisition unit 103.

The display unit 106 is formed of a CRT (cathode ray tube) or an LCD (liquid crystal display), and displays the B mode image.

The input unit 107 is formed of a keyboard or track ball, and inputs with an operation input signal from the operator. For example, an operation input signal for selecting the B mode display is input from the input unit 107. Then, those information is transmitted to the control unit 108. Also, the input unit 107 also has a start timing key that starts the amplitude voltage control means 40 that will be described later, and starts the change of the amplitude voltage of the drive voltage waveform.

The control unit 108 controls the operation of the above respective portions of the ultrasonic imaging apparatus on the basis of the operation input signal that has been input from the input unit 107, or program or data which have been stored in advance.

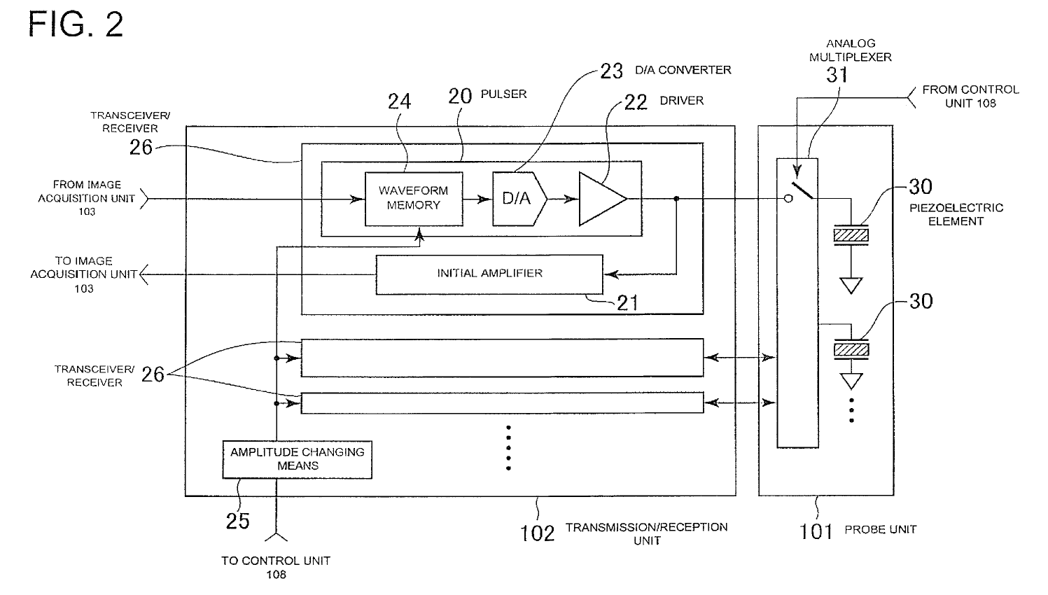

FIG. 2 is a block diagram showing the detailed configuration of the transmission/reception unit 102 and the probe unit 101. The transmission/reception unit 102 includes transceiver/receiver 26 and amplitude change means 25 which are driven almost at the same time. The transceiver/receiver 26 includes the pulser 20 and the initial amplifier 21. The probe 101 includes an analog multiplexer 31 that selectively and electrically connects the piezoelectric elements 30 that are arranged in the array, the piezoelectric elements 30, and the transceiver/receiver 26 with each other.

The pulser 20 includes a driver 22, a D/A converter 23, and a waveform memory 24. The waveform memory 24 is a memory that stores the drive voltage waveform information of the piezoelectric element 30. The drive voltage waveform information is continuously read from the waveform memory 24 and then input to the D/A converter 23.

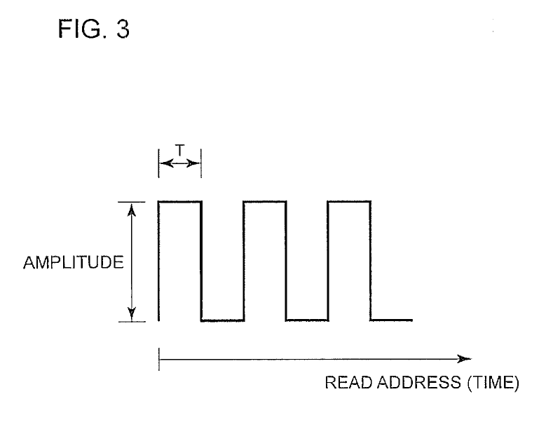

FIG. 3 schematically shows an example of the drive voltage waveform information that has been recorded in the waveform memory 24 with the axis of ordinate as the amplitude and the axis of abscissa as the address order of the read. In FIG. 3, there is shown an example of a burst waveform in which three rectangular pulses of a pulse width T having the same amplitude are arranged.

Returning to FIG. 2, the D/A converter 23 converts the input drive voltage waveform information to an analog signal, and inputs the signal to the driver 22. The driver 22 amplifies the input analog drive voltage waveform, and applies the amplified waveform to the piezoelectric elements 30 of the probe 101. As a result, the piezoelectric element 30 generates the same ultrasonic pulse as that of the drive voltage waveform.

The piezoelectric elements 30 are connected to the driver 22 through the analog multiplexer 31. The analog multiplexer 31 turns on/off a switch that allows the analog signal to pass according to a control signal from the control unit 108, and conducts the selection and electronic scanning of the piezoelectric elements 30 that are arranged in an array that is included in the probe 101.

The reflection ultrasonic pulse train from the object to be detected that has been received in the piezoelectric elements 30 is input to the initial amplifier 21 of the transmission/reception unit 102, and then transmitted to the image acquisition unit 103 after amplification.

An amplitude change means 25 changes the amplitude of the drive voltage waveform information that has been recorded in the waveform memory 24 according to the control signal from the control unit 108. As a result, the amplitude voltage of the drive voltage waveform that is output from the driver 22 is similarly changed, and therefore the sound pressure amplitude of the ultrasonic pulse that is output from the piezoelectric element 30 depends on the amplitude voltage of the drive voltage waveform.

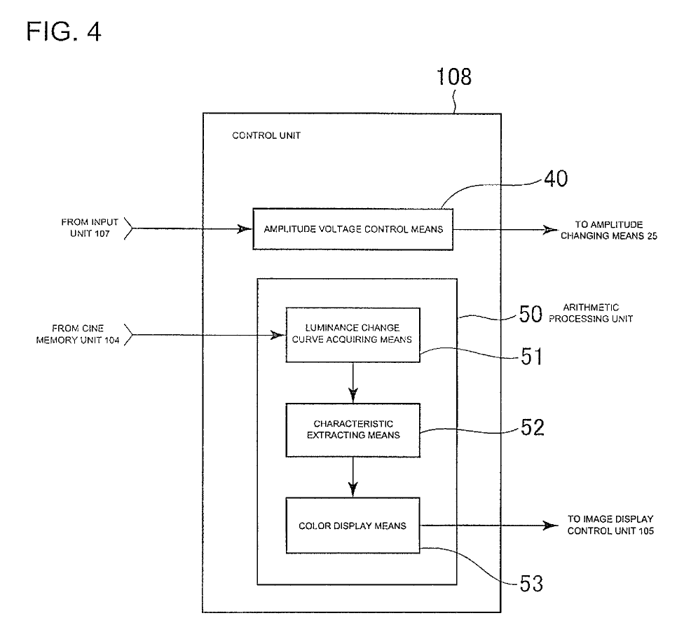

FIG. 4 is a functional block diagram showing the functional configuration of the control unit 108. The control unit 108 includes the amplitude voltage control means 40 and the arithmetic processing unit 50. The arithmetic processing unit 50 includes luminance change curve acquiring means 51, characteristic extracting means 52, and color display means 53.

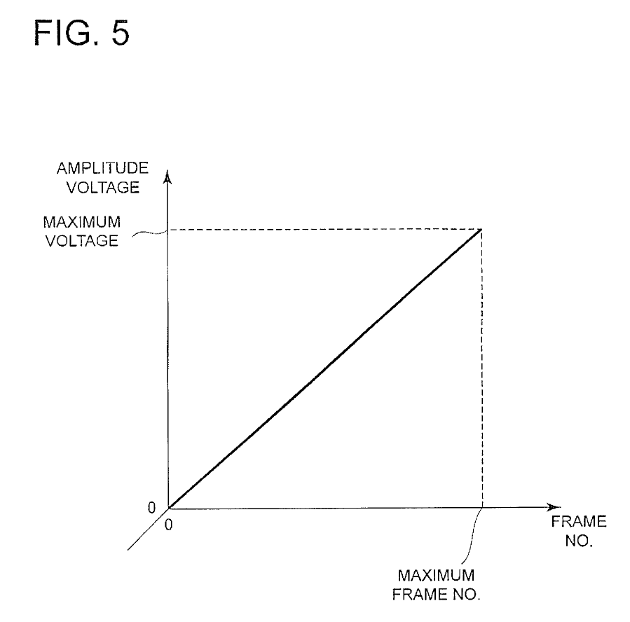

The amplitude voltage control means 40 starts a change in the amplitude voltage according to the start timing key information for starting the change in the amplitude voltage from the input unit 107. The amplitude voltage control means 40 transmits the amplitude information to the amplitude change means 25 of the transmission/reception unit 102 in synchronism with the start timing information, and changes the amplitude voltage of the drive voltage waveform that is applied to the piezoelectric elements 30. The change in the amplitude voltage is uniformly increased from zero volt according to the imaging order of the frame with the frame that forms a piece of the tomographic image information as a unit. FIG. 5 is an explanatory diagram showing a change in the amplitude voltage which is caused by the amplitude voltage control means 40 with the axis of ordinate as the amplitude voltage and the axis of abscissa as the frame number after a start signal has been input. The amplitude voltage of the drive voltage waveform is increased in proportion to the number of frames. The maximum value of the amplitude voltage is a voltage that produces a sufficient sound pressure for destroying the contrast agent in the object to be detected. Also, the control unit 108 sequentially stores the imaged tomographic image information in the cine memory unit 104 after the start timing key information that starts the change in the amplitude voltage has been input to the control unit 108.

The arithmetic processing unit 50 acquires the luminance change curve of the inspection region in each of the inspection regions that section the region of the tomographic image from the tomographic image information with the use of plural pieces of tomographic image information that are different from each other in the amplitude voltage of the drive voltage waveform that has been stored in the cine memory unit 104. The arithmetic processing unit 50 also calculates and displays the characteristic parameter in the inspection region from the above curve. The detailed functions and operation of the luminance change curve acquiring means 51, the characteristic extracting means 52, and the color display means 53 which are included in the arithmetic processing unit 50 will be described in association with the following description of the operation of the ultrasonic imaging apparatus 100.

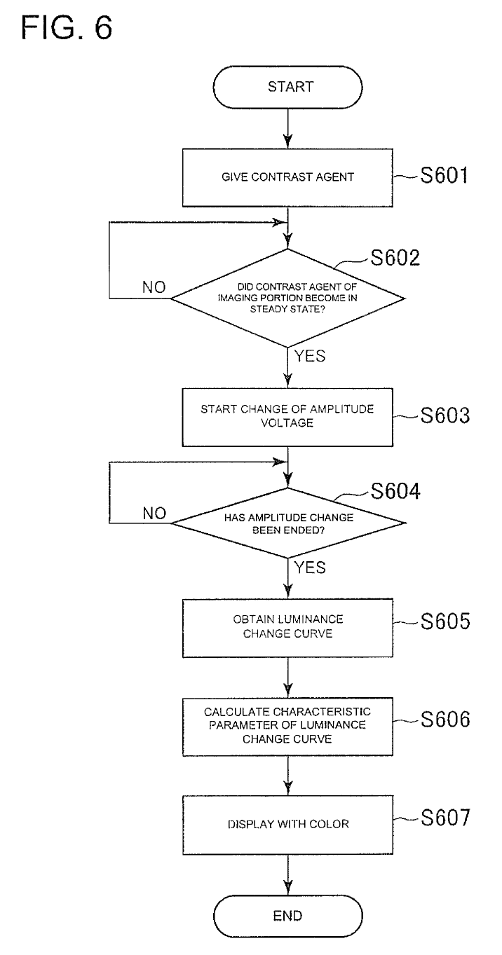

Subsequently, the operation of the ultrasonic imaging apparatus 100 according to this embodiment will be described with reference to FIG. 6. FIG. 6 is a flow chart showing the operation of the ultrasonic imaging apparatus 100. First, the operator gives the object to be detected the contrast agent (Step S601). The contrast agent is injected into the venation of the object to be detected, and then circulated in the entire body of the object to be detected through the artery.

Thereafter, the operator brings the probe unit 101 in close contact with an intended imaged portion of the object to be detected, observes the B mode image of the ultrasonic imaging apparatus 100, and confirms that tho contrast agent flows into the artery. Then, the operator makes the contrast agent flow into the tissue portion of the imaged portion, and determines whether the contrast agent is evenly distributed into the imaged portion, and becomes in a steady state, or not (Step S602). In the case where the contrast agent of the imaged portion is not in the steady state (no in Step S602), the operator observes the repetitive B mode image, and waits for the steady state. Also, in the case where the contrast agent of the imaged portion is in the steady state (yes in Step S602), the operator depresses the start timing key of the input unit 107, and starts the change in the amplitude voltage of the drive voltage waveform (Step S603). In this example, the amplitude voltage control means 40 conducts imaging while increasing the amplitude voltage of the drive voltage waveform from the zero voltage in proportion to the order to the frames to be imaged as shown in FIG. 5. The imaging frame number is reset at the same time when the start timing key is depressed, starts from zero, and sequentially increases to the maximum frame number where the amplitude becomes the maximum voltage. In this situation, the B mode image information in each of the imaged frames is stored in the cine memory unit 104.

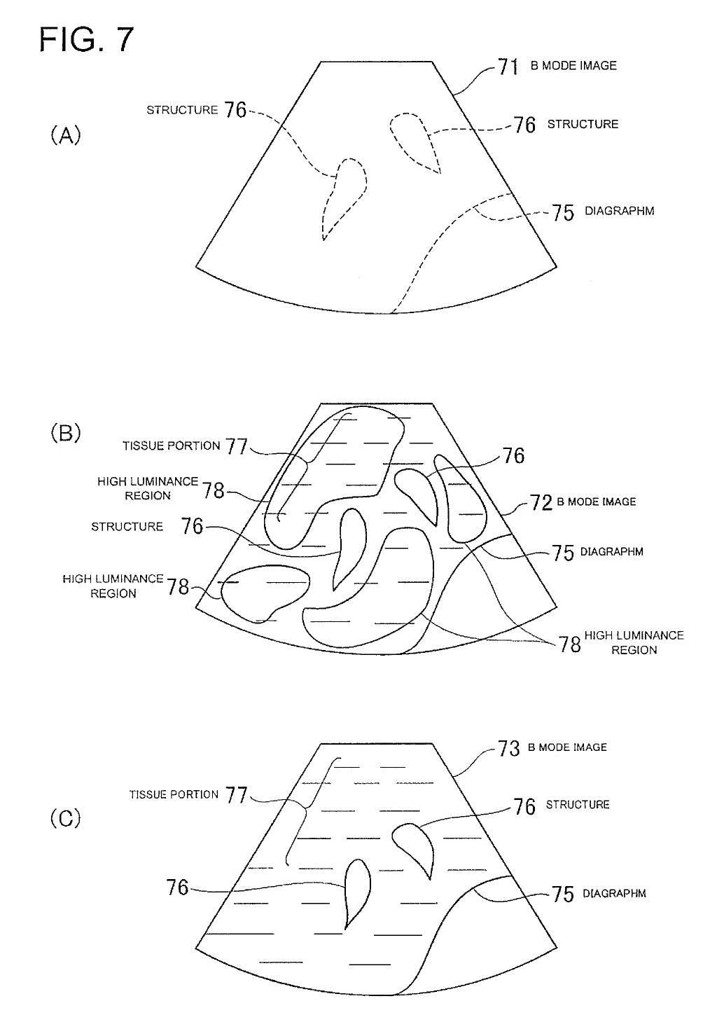

FIGS. 7(A), 7(B), and 7(C) are explanatory diagrams schematically showing the B mode image information that has been stored in the cine memory unit 104. At first, in the case where the amplitude voltage of the drive voltage waveform is close to the zero volt, the reflection ultrasonic pulse train from the object to be detected is not observed, and the B mode image to be acquired becomes a dark image having no luminance at all. Then, the reflection ultrasonic pulse train from the object to be detected gradually starts to be observed while the amplitude voltage of the drive voltage waveform gradually increases. FIG. 7(A) schematically shows the B mode image 71 of the above appearance, and the high luminance portions such as a diaphragm 75 and structures 76 of the tissue indicated by broken lines in the figure gradually starts to be visible.

Then, as the amplitude voltage of the drive voltage waveform further increases, the clear B mode image of the diaphragm 75, the strictures 76, and the tissues 77 are observed in addition to the B mode image of the reflection ultrasonic pulse train due to the enlargement/reduction motion of the contrast agent that has penetrated the tissues. FIG. 7(B) schematically shows the B mode image 72 of the above appearance, from which the diaphragm 75, the structures 76, and the tissues 77 that are indicated by broken lines, which are arranged in the horizontal direction are clearly visualized, and the high luminance region 78 having the contrast agent exists in the tissues 77.

Then, as the amplitude of the drive voltage waveform further increases, the contrast agent that has penetrated the tissues is destroyed, and the high luminance region 78 disappears. FIG. 7(C) schematically shows the B mode image 73 of the above appearance, in which the high luminance region 78 disappears, and only the clear B mode images of the structures 76 and the tissues 77 remain. The timings of the occurrence of the high luminance region 78 and the disappearance of the high luminance region 78 which occur due to the existence of the contrast agent 76 are different depending on the amplitude of the sound pressure of the ultrasonic pulse that is irradiated on the object to be detected, the peripheral environments where the contrast agent is positioned in the imaging section, for example, the existence of a tumor.

Thereafter, returning to FIG. 6, the control unit 108 determines whether the frame number is the maximum frame number, and the change in the amplitude voltage of the drive voltage waveform has been completed, or not (Step S604). In the case where completion is not made (no in Step S604), the control unit 108 waits for the completion of the change, and in the case where the completion is made (yes in Step S604), the luminance change curve is obtained by the luminance change curve acquiring means 51 with the use of the tomographic image information that has been stored in the cine memory unit 604 (Step S605).

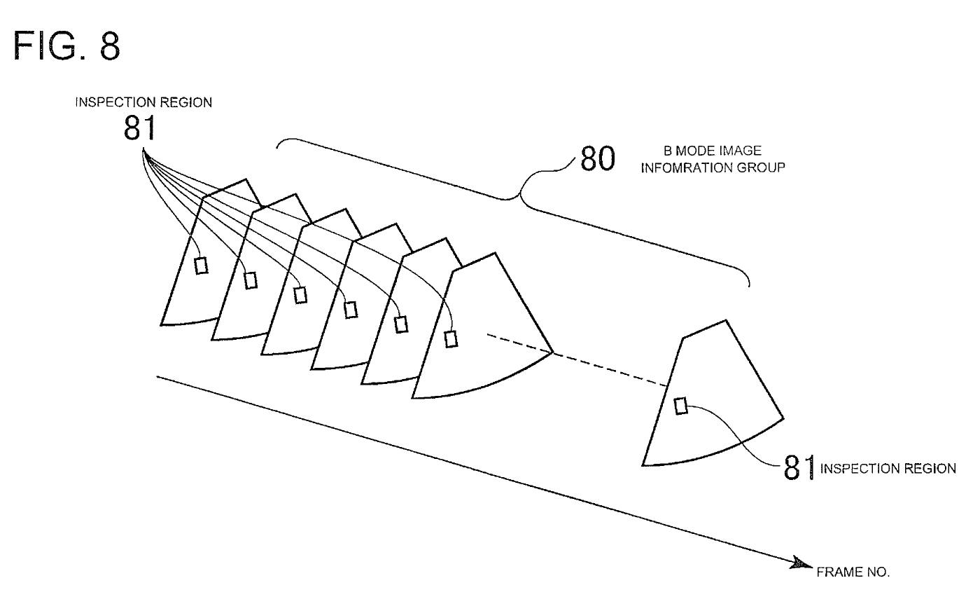

FIG. 8 schematically shows the B mode image information group 80 where the amplitude voltage is increased in proportion to the acquisition No. of the frame which is stored in the cine memory unit 104. The frame acquisition No. is indicated by frame number. The B mode image information group 80 consists of plural pieces of B mode image information where the amplitude voltage of the drive voltage waveform, in other words, the sound pressure amplitude of the ultrasonic pulse which is applied to the object to be detected, continuously changed from zero to a given value in proportion to the frame number.

The luminance change curve acquiring means 51 sections the B mode image into the plural inspection regions, and obtains the luminance change curve in each of the inspection regions with the use of the B mode image information group 80. The inspection region 81 shown in FIG. 8 is one of the sectioned inspection regions. The luminance change curve acquiring means 51 calculates the average luminance value of pixels that are included in the inspection region 81 with respect to the B mode image information of all frame numbers which arc different in the sound pressure, and obtains the luminance change curve.

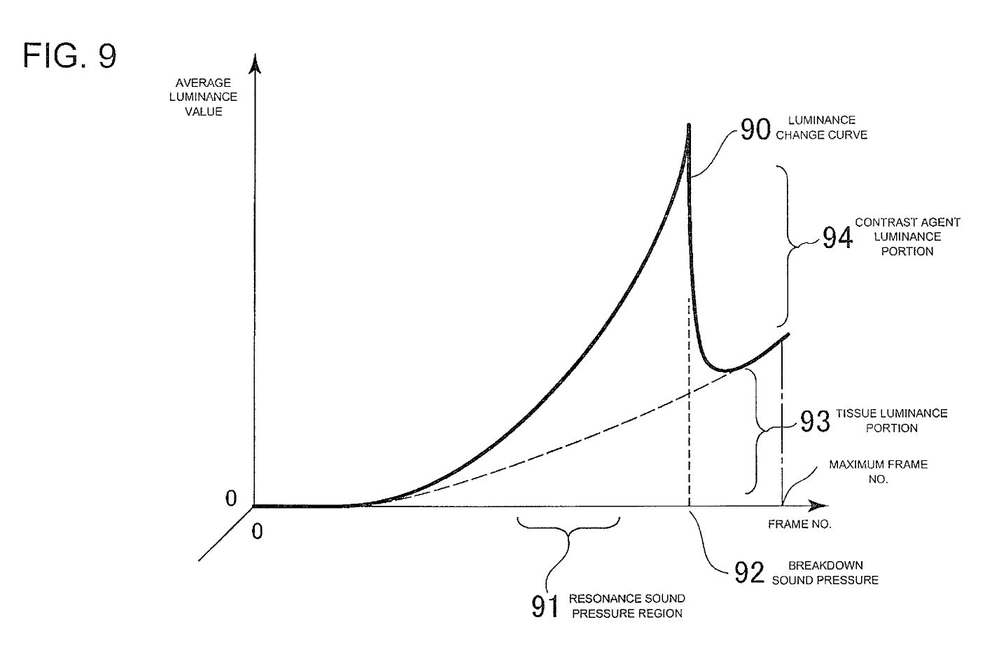

FIG. 9 is an explanatory diagram showing an example of an acquired luminance change curve 90. The luminance change curve 90 consists of a tissue luminance portion 93 having the luminance values of the tissues, and a contrast agent luminance portion 94 having the luminance values of the contrast agent. In the region of the small frame number where the sound pressure is low, the average luminance value is increased substantially in proportion to the sound pressure. In this example, in the resonance sound pressure region 91 of the resonance state in which the contrast agent exists, and air bubbles that constitute the contrast agent conduct the enlargement/reduction motion, the contrast agent luminance portion 94 rapidly increases, and the luminance change curve 90 also increases. Also, in the breakdown sound pressure 92 where the air bubbles that constitute the contrast agent are destroyed, the contrast agent luminance portion 94 rapidly decreases, and the luminance change curve 90 also decreases.

The luminance change curve acquiring means 51 obtains the luminance change curve 90 in all of the inspection regions that cover the B mode image.

Thereafter, returning to FIG. 6, the control unit 108 calculates the characteristic parameter of the luminance change curve 90 by the aid of the characteristic extracting means 52 (Step S606). In this example, the resonance sound pressure region 91 and the frame number of the breakdown sound pressure 92 exist as the characteristic parameter of the luminance change curve 90.

The characteristic extracting means 52 calculates the resonance sound pressure region 91 and the frame number of the breakdown sound pressure 92 from the luminance change curve 90. In the case of obtaining the resonance sound pressure region 91, the characteristic extracting means 52 sets a threshold value that is experimentally determined in the average luminance value by the aid of the first characteristic extracting means, and sets the frame number where the luminance change curve 90 exceeds the threshold value as a representative value of the resonance sound pressure region 91. Also, in the case of obtaining the breakdown sound pressure 92, the characteristic extracting means 52 obtains the maximum value of the luminance change curve 90 by the aid of the second characteristic extracting means, and sets the frame number where the maximum value exists as a value of the breakdown sound pressure 92.

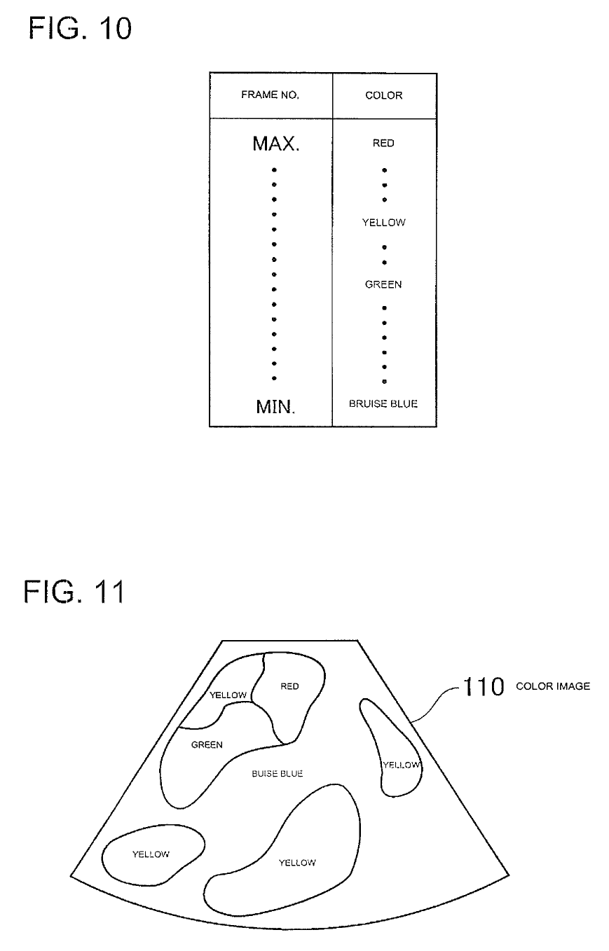

Thereafter, the control unit 108 relates the frame numbers of the resonance sound pressure region 91 and the breakdown sound pressure 92 to the hues by the aid of the color display means 53, and displays the resonance sound pressure region 91 or the breakdown sound pressure 92 in each of the inspection regions 81 on the same display screen as the B mode image with colors (Step S607), and completes this processing. An example in which the frame number is subjected to color coding is shown in FIG. 10. FIG. 10 is a table showing the frame numbers and the related hues, which are set in the color display means 53 in advance. In the table, the maximum value and the minimum value of the frame numbers are obtained, and the frame numbers between the maximum value and the minimum value are associated with colors of red to bruise blue which are continuous in the visible region. The color display means 53 obtains the hues in each of the inspection regions with the use of the table. Then, the color display means 53 displays an image having the same configuration as that of the B mode image on the display unit 106 through the image display control unit 105, and displays the hues corresponding to the frame numbers at a location corresponding to each of the inspection regions in the image.

FIG. 11 is an explanatory diagram schematically showing the color image 110 that is displayed on the display unit 106. The color image 110 has the same configuration as the B mode images 71 to 73, and becomes images that are sectioned in the inspection region having the colors of red to bruise blue. As a result, the change and the distribution of the resonance sound pressure region 91 or the breakdown sound pressure 92 in the mode images 71 to 73 where the intended imaged region has been extracted is visualized and can be easily grasped.

As described above, in this embodiment, when the contrast agent penetrates the tissues of the imaged portion to provide the steady state, the amplitude voltage of the drive voltage waveform is increased in proportion to the frame number indicative of the frame acquisition number from zero volt by the aid of the amplitude voltage control means 40 to conduct imaging. Then, the frame numbers corresponding to the resonance sound pressure region where the contrast agent conducts the enlargement/reduction motion and the breakdown sound pressure where the contrast agent is destroyed are obtained by the aid of the luminance change curve acquiring means 51 and the characteristic extracting means 52. Color coating is conducted on those frame numbers by the color display means 53, and the distribution of the resonance sound pressure region or the breakdown sound pressure is displayed with colors. As a result, it is possible to easily detect and grasp the resonance sound pressure region or the distribution within the imaging section of the breakdown sound pressure.

Also in this embodiment, when the contrast agent penetrates the tissues to come to the steady state, the amplitude voltage of the drive voltage waveform is changed. Alternatively, it is possible that after the contrast agent has been given to the venation of the object to be detected, the contrast agent that is first circulated in the artery through a heart is changed while the amplitude voltage of the drive voltage waveform is synchronized with the chapter function of the cine capture means to obtain the resonance sound pressure region 91 and the breakdown sound pressure 92. The cine capture means synthesizes the maximum projection value image where the images at the same imaging positions that temporally change are superimposed on each other by the aid of the MIP (maximum intensity projection).

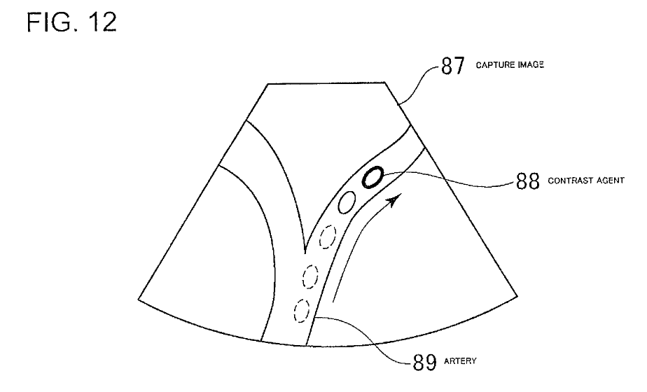

FIG. 12 is an explanatory diagram schematically showing the appearance of the contrast agent that flows in the artery which is observed in the capture image in the case where the amplitude voltage of the drive voltage waveform changes in synchronization with the capture as shown in FIG. 5. The capture image 87 extracts the contrast agent 88 that travels in the artery 89. The contrast agent 88 is extracted in the form of particles, and travels along the flow of a bloodstream indicated by an arrow in the figure in each of the captures. With a change in the amplitude voltage of the drive voltage waveform, the average luminance value of the region where the contrast agent 88 exists changes substantially as shown in FIG. 9. In other words, the contrast agent 88 travels while the average luminance value increases, and the average luminance value becomes maximum when the contrast agent 88 reaches the breakdown sound pressure, and thereafter the average luminance value disappears. Accordingly, the control unit 108 obtains the luminance change curve 90 on the basis of a change in the average luminance value of the contrast agent 88 in each of the captures, and is capable of obtaining the resonance sound pressure region 91 and the breakdown sound pressure 92 of the contrast agent.

Also, in this embodiment, the change in the amplitude voltage of the drive voltage waveform starts due to the input of the start timing key of the input unit 107. Alternatively, it is possible that the input unit 107 is provided with an administration timing key and a delay time set key, and the administration timing key is input at the time of giving the contrast agent, and the change in the amplitude voltage of the drive voltage waveform can start after a delay time that is set from the delay time set key of the input unit 107 in advance.

Also, in this embodiment, the resonance sound pressure region or the breakdown sound pressure of the contrast agent is obtained as the characteristic parameter of the luminance change curve 90. Alternatively, it is possible to obtain the linear characteristic between the luminance value and the sound pressure (frame number) according to a slope of the luminance change curve 90.

Also, in this embodiment, the sound pressure of the ultrasonic pulse is changed in accordance with the frame acquisition number. Alternatively, the number of bursts in the case of conducting the frequency or burst drive of the ultrasonic pulse can be changed according to the frame acquisition number.

Many widely different embodiments of the invention may be configured without departing from the spirit and the scope of the present invention. It should be understood that the present invention is not limited to the specific embodiments described in the specification, except as defined in the appended claims.

1. An ultrasonic imaging apparatus, comprising:
a probe that transmits configured to transmit an ultrasonic pulse to an object to be detected and receive a reflection ultrasonic pulse train from the object to be detected;
a sound pressure amplitude control device configured to automatically change a sound amplitude of the ultrasonic pulse according to an acquisition number of a frame indicative of an order of the acquisition, with one frame including a repetition of the transmission and the reception which acquires a piece of tomographic image information of the object to be detected as a minimum change unit; and
a display unit configured to display the tomographic image information.

2. The ultrasonic imaging apparatus according to claim 1, wherein the object to be detected is given a contrast agent.

3. The ultrasonic imaging apparatus according to claim 1, wherein said sound pressure amplitude control device is configured to change the sound pressure amplitude in proportion to the acquisition number of the frame.

4. The ultrasonic imaging apparatus according to claim 1, wherein said sound pressure amplitude control device comprises:
a transmission/reception unit configured to form a drive voltage waveform which generates the ultrasonic pulse in a piezoelectric element of the probe; and
an amplitude voltage control device configured to automatically change the amplitude voltage of the drive voltage waveform according to the acquisition number.

5. The ultrasonic imaging apparatus according to claim 1, further comprising an image memory configured to record the tomographic image information.

6. The ultrasonic imaging apparatus according to claim 4, further comprising an input unit configured to input the control information of said amplitude voltage control device.

7. The ultrasonic imaging apparatus according to claim 6, wherein said input unit comprises a start timing key configured to start a change in the amplitude voltage.

8. The ultrasonic imaging apparatus according to claim 6, wherein said input unit comprises an administration timing key configured to input an administration timing when giving the object to be detected the contrast agent, and a delay time input key configured to input a delay time from the administration timing.

9. The ultrasonic imaging apparatus according to claim 8, wherein said amplitude voltage control device is configured to start the change in the amplitude voltage at a time delayed by the delay time from the administration timing.

10. The ultrasonic imaging apparatus according to claim 4, wherein said amplitude voltage control device is configured to change the magnitude of the amplitude voltage in an increasing direction from a value of zero.

11. The ultrasonic imaging apparatus according to claim 1, further comprising an arithmetic processing unit configured to arithmetically process the tomographic image information.

12. The ultrasonic imaging apparatus according to claim 11, wherein said arithmetic processing unit further comprises a luminance change curve acquiring device configured to obtain a luminance change curve indicative of a change in an average luminance value of a detection region due to the frame in each of the detection regions that section the tomographic image of the frame by using a plurality of frames on the sane section which are different in the amplitude voltage of the drive voltage waveform from each other.

13. The ultrasonic imaging apparatus according to claim 12, wherein said arithmetic processing unit further comprises a first characteristic extracting device configured to obtain a frame number of the average luminance value having a threshold value when the average luminance value of the luminance change curve passes through the threshold value.

14. The ultrasonic imaging apparatus according to claim 12, wherein said arithmetic processing unit further comprises a second characteristic extracting device configured to obtain a frame number of a maximum value of the luminance change curve.

15. The ultrasonic imaging apparatus according to claim 13, wherein said arithmetic processing unit further comprises a color display device having a hue table that relates the frame number to the hue, wherein said color display device is configured to obtain the hue of the detection region on the basis of the hue table.

16. The ultrasonic imaging apparatus according to claim 15, wherein said display unit is configured to display a color image with a color of the hue in the detection region of the tomographic image.

17. The ultrasonic imaging apparatus according to claim 11, wherein said arithmetic processing unit comprises a capture device configured to obtain the maximum value of pixel values at the same pixel position of the tomographic images of the plurality of frames by using the plurality of frames on the same section which are different in the amplitude voltage of the drive voltage waveform from each other to thereby synthesize maximum projection value image information that is comprised of the maximum value.

18. The ultrasonic imaging apparatus according to claim 17, wherein said display unit is configured to display the maximum projection value image information.

19. An ultrasonic imaging method comprising:
transmitting an ultrasonic pulse to an object to be detected; and
receiving a reflection ultrasonic pulse train from the object to be detected; and
changing automatically a sound amplitude of the ultrasonic pulse according to an acquisition number of a frame indicative of an order of the acquisition, with one frame including a repetition of the transmission and the reception which acquires a piece of tomographic image information of the object to be detected as a minimum change unit.

20. The ultrasound imaging method according to claim 19, further comprising displaying the tomographic image information.
